(19) United States
(12) Patent Application Publication
(10) Pub. No.: US 2007/0225615 A1
(43) Pub. Date: Sep. 27, 2007

(54) Guidewire controller system

(75) Inventors: Victor Chechelski, Mountain View, CA (US); Gerardo V. Noriega, Mountain View, CA (US); Keith Riordan, Saratoga, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: ReVascular Therapeutics Inc., Sunnyvale, CA (US)

(21) Appl. No.: 11/388,251

(22) Filed: Mar. 22, 2006

(51) Int. Cl. A61M 25/00; A61B 17/00

(52) U.S. Cl. 600/585; 606/1

(57) ABSTRACT

A guidewire controller system includes a guidewire device and a control unit. The guidewire device has an axial lumen and a drive shaft extending through the axial lumen. The control unit is coupled to the guidewire device. The control unit has a processor which produces a variable sound in response to a load measurement on the drive shaft. The load may be measured by a change in current in a motor which drives the shaft. This change in current is then converted to a frequency for variable sound.

### BACKGROUND OF THE INVENTION

The present invention is generally related to medical systems and methods. More specifically, the present invention relates to a guidewire controller system and method for providing control feedback during crossing stenosis, partial occlusions, or total occlusions in a patient's body, such as in a body or vessel lumen.

Cardiovascular disease frequently arises from the accumulation of atheromatous material on the inner walls of vascular lumens, particularly arterial lumens of the coronary and other vasculature, resulting in a condition known as atherosclerosis. Atheromatous and other vascular deposits restrict blood flow and can cause ischemia which, in acute cases, can result in myocardial infarction or a heart attack. Atheromatous deposits can have widely varying properties, with some deposits being relatively soft and others being fibrous and/or calcified. In the latter case, the deposits are frequently referred to as plaque. Atherosclerosis occurs naturally as a result of aging, but may also be aggravated by factors such as diet, hypertension, heredity, vascular injury, and the like.

Atherosclerosis can be treated in a variety of ways, including drugs, bypass surgery, and a variety of catheter-based approaches which rely on intravascular widening or removal of the atheromatous or other material occluding the blood vessel. Particular catheter-based interventions include angioplasty, atherectomy, laser ablation, stenting, and the like. For the most part, the catheters used for these interventions must be introduced over a guidewire, and the guidewire must be placed across the lesion prior to catheter placement. Initial guidewire placement, however, can be difficult or impossible in tortuous regions of the vasculature. Moreover, it can be equally difficult if the lesion is total or near total, i.e. the lesion occludes the blood vessel lumen to such an extent that the guidewire cannot be advanced across the lesion.

To overcome this difficulty, forward-cutting atherectomy catheters have been proposed. Such catheters usually can have a forwardly disposed blade (U.S. Pat. No. 4,926,858) or rotating burr (U.S. Pat. No. 4,445,509). While effective in some cases, these catheter systems, even when being advanced through the body lumen with a separate guidewire, have great difficulty in traversing through the small and tortuous body lumens of the patients and reaching the target site.

Guidewires for crossing occlusions or stenoses which can access small, tortuous regions of the vasculature and which can remove atheromatous, thrombotic, and other occluding materials from within blood vessels are described in U.S. patent application Ser. No. 11/236,703, filed Sep. 26, 2005, assigned to the assignee of the present application and incorporated herein by reference. While such guidewire devices successfully pass through partial occlusions, total occlusions, or stenosis, and are able to macerate blood clots or thrombotic material, further improvements would be advantageous.

### BRIEF SUMMARY OF THE INVENTION

The present invention relates to guidewire controller systems and methods for providing control feedback during crossing stenosis, partial occlusions, or total occlusions in a patient's body, such as in a body or vessel lumen. The devices for removing occlusive material and passing through occlusions, stenosis, thrombus, plaque, calcified material, and other material in a neuro, coronary, and peripheral body lumens generally include an elongate member, such as a hollow guidewire device, that is advanced through a blood vessel lumen and positioned adjacent the occlusion or stenosis. An occlusive material (e.g., plaque) removal assembly is positioned at or near a distal tip of the hollow guidewire to create an opening in the occlusion. The plaque removal assembly generally comprises a drive shaft having a distal tip that is oscillated, axially reciprocated (e.g., pecking), rotated and/or vibrated and advanced from within an axial lumen of the hollow guidewire. Once the guidewire has reached the lesion, the guidewire with the exposed oscillating, axially reciprocating, rotating and/or vibrating drive shaft may be advanced into the lesion (or the guidewire may be in a fixed position and the drive shaft may be advanced) to create or form a path forward of the hollow guidewire in the occlusion or stenosis.

Advantageously, the guidewire controller systems of the present invention provide a control unit coupled to the guidewire device. The control unit has a processor which produces a variable sound in response to a load measurement on the drive shaft, particularly during advancement of the distal tip in the occluded vessel lumen. The load measurement comprises a change in current in a motor which drives the shaft. It will be appreciated however that the load may be measured in a variety of other ways, as for example measuring a change in voltage, amperage, or other electrical signals related to the drive shaft motor, such as monitoring the change of rotational speed of the drive shaft via an encoder within the motor. The drive motor is generally mechanically attachable to a proximal end of the drive shaft to move (e.g., oscillating, axially translating, reciprocating, rotating, vibrating) the drive shaft and distal tip.

This change in current, which accurately measures the load on the drive shaft, is then converted to a frequency for variable sound. The sound will generally comprise a pitch or tone which is proportional to the measured load on the motor, wherein a relationship between the sound and the load measurement is substantially linear. For example, as the measured load or resistance encountered increases, the pitch or tone of the sound increases. A speaker is electrically coupled to the processor in the control unit for emitting the sound. Further, an audio amplifier may be electrically coupled between the processor and the speaker in the control unit to amplify the sound prior to emission from the speaker.

The guidewire device, which is described in more detail in co-pending U.S. patent applicatioh Ser. No. 11/236,703, comprises an elongate hollow deflectable body having an axial lumen. The drive shaft preferably comprises an oscillatory core element which is movably receivable within the axial lumen. A handle may further be coupled to a proximal end of the guidewire device. The motor preferably resides within a distal end of the guidewire handle and is mechanically secured to avoid any oscillatory or axial movement during operation. Typically, the drive motor is coupled to the control unit via wire leads or cables. The electronic circuitry in the control unit (e.g., processor) controls activation of the motor to oscillate (e.g., polarity, period, time), axially translate, reciprocate, rotate and/or vibrate the drive shaft besides measuring loads and producing variable sounds associated therewith. It will be appreciated that the control unit may optionally be positioned with the drive motor within the handle component of the guidewire device.

In another aspect of the present invention, methods for providing control feedback during crossing of an occlusion or stenosis within a vessel lumen are provided. A guidewire device, as described above, is positioned into the vessel lumen adjacent the occlusion or stenosis. A drive shaft is activated within an axial lumen of the guidewire device. A level of load on the drive shaft is measured. In response to the load measurement, a variable sound is produced.

Measuring a load comprises measuring a change in current in a motor which drives the shaft. Typically, the load on the drive motor is expressed in milliamps and varies according to the resistance encountered by the drive shaft, particularly its distal tip, in the occluded vessel lumen. The load on the motor may be detected through the measurement of voltage across a known resistor which is directly proportional to the current flowing through the resistor. The resistor may have a resistance in a range from about 0.1 ohms to about 10 ohms. For example, two 1 ohm resistors may be provided for an oscillatory drive shaft, one resistor for each direction of the oscillatory drive motor. The amperage related voltage is then compared to a reference voltage, which may be in a range from about 0.2 volts to about 1.0 volts, as for example 0.53 volts.

A variable sound is then produced by converting the change in current (i.e., the difference between the measured load and the reference voltage) to a frequency for sound. The sound comprises a pitch or tone which is substantially linearly proportional to the measured load on the motor. For example, the larger the difference between the measured load and the reference voltage, the larger the change in the pitch or tone of the sound. The different audible tones of the sound are noticeable due the change of the frequency of the signal generated by change in the electrical signal of the load measurement. Hence, the sound, which is expressed in hertz, varies according to the resistance encountered by the drive shaft as measured by the current change in the drive motor.

The sound may be in a range from about 30 hertz to about 10,000 hertz, preferably in a range from about 50 hertz to about 3,000 hertz and indicate a variety of load conditions within the vessel lumen. In particular, the control unit may provide higher to lower pitches depending on the resistance encountered by the motor through the drive shaft distal tip. For example, the variable sound may have an increasing or high pitch or tone so as to indicate a high level of occlusion in the vessel lumen. In this instance, the drive shaft distal tip is encountering high resistance so as to indicate a high level of calcification inside the vessel lumen. Alternatively, the variable sound may have a decreasing or low pitch or even no pitch (e.g., no sound) so as to indicate that the guidewire device is outside the vessel lumen and within sub-intimal tissue. In this instance, the drive shaft distal tip encounters much less resistance outside the vessel lumen as opposed to the high resistance encountered during crossing a hard calcified occlusion inside the vessel lumen. Accordingly, the sound emitted may have a much lower intensity or may not even be noticeably present. Still further, the variable sound may have a constant pitch that indicates that the guidewire device is non-operational. In this instance, the zero or no load measurement may indicate a break or fracture in the drive shaft or that the drive shaft distal tip is encountering no resistance, resulting in no change in the sound being emitted. In addition, the physician may face tactile mechanical resistance when attempting to advance the distal tip of the device. The electronic circuitry (e.g., processor) in the control unit may further automatically disable the guidewire device in this situation for safety purposes.

The methods may further comprise emitting the sound from a speaker. Optionally, the sound may be amplified prior to emission from the speaker via an audio amplifier which may additionally be adjustable. As described above, activating may comprise oscillating the drive shaft. Oscillation of the drive shaft may further comprise changing polarity after a period of time in a range from about 0.3 seconds to about 1.2 seconds, preferably in a time range of about 0.7 seconds. The activating, measuring, producing, and changing polarity steps are carried out by electronic circuitry (e.g., processor) in the control unit.

A further understanding of the nature and advantages of the present invention will become apparent by reference to the remaining portions of the specification and drawings.

### BRIEF DESCRIPTION OF THE DRAWINGS

The following drawings should be read with reference to the detailed description. Like numbers in different drawings refer to like elements. The drawings, which are not necessarily to scale, illustratively depict embodiments of the present invention and are not intended to limit the scope of the invention.

FIG. 1 illustrates an exemplary guidewire controller system constructed in accordance with the principles of the present invention.

FIG. 2 illustrates an exploded view of a distal end portion of the guidewire device of FIG. 1 comprising a drive shaft disposed within a hollow, deflectable body.

FIG. 3 is a simplified block diagram illustrating the controller system of the present invention.

FIG. 4 is a simplified flow diagram illustrating a method for providing control feedback during crossing of an occlusion or stenosis within a vessel lumen in accordance with the principles of the present invention.

FIGS. 5 through 7 illustrate exemplary electrical schematic drawings for electronics that can be used in an embodiment of the present invention.

### DETAILED DESCRIPTION OF THE INVENTION

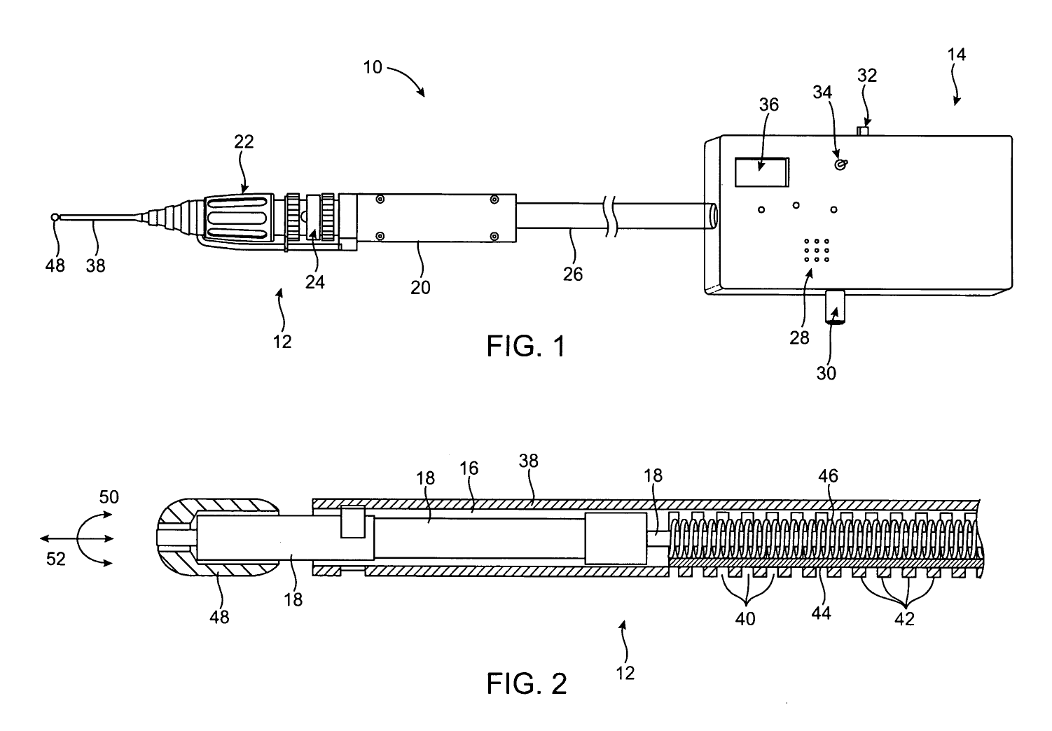

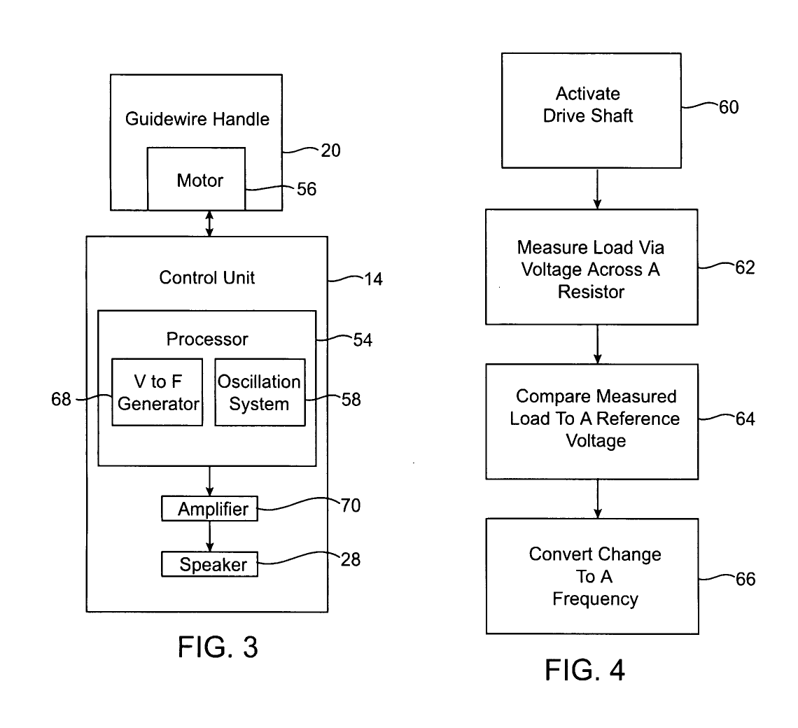

Referring now to FIG. 1, an exemplary guidewire controller system 10 constructed in accordance with the principles of the present invention is illustrated. The guidewire controller system 10 includes a guidewire device 12 and a control unit 14. The guidewire device 12 has an axial lumen 16 and a drive shaft 18 extending through the axial lumen 16, as best shown in FIG. 2. A handle 20 having a torquer knob 22 to torque the guidewire device 12 and a deflection wheel 24 to deflect the guidewire device 12 may further be coupled to a proximal end of the guidewire device 12. The control unit 14 is coupled to the guidewire handle 20 via wire leads or cables 26. The control unit 14 includes a speaker 28, a volume control knob 30 which may adjust audio amplification, a main on/off power supply switch 32, a momentary switch 34, and a timer liquid crystal display (LCD) 36. The control unit may have a length in a range from about 5 cm to about 25 cm, preferably 17 cm, a width in a range from about 5 cm to about 12 cm, preferably 8.5 cm, and a depth in a range from about 1 cm to about 8 cm, preferably 3.5 cm. It will be appreciated that the above depictions are for illustrative purposes only and do not necessarily reflect the actual shape, size, or dimensions of the controller system 10. This applies to all depictions hereinafter.

Referring now to FIG. 2, the guidewire device 12 of the present invention has steerability, deflectability, flexibility, pushability, and torqueability to be advanced through the tortuous blood vessel without the use of a separate guidewire or other guiding element. Additionally, the guidewire device 12 may be sized to fit within an axial lumen of a distal support or access catheter system (not shown), which is described in more detail in U.S. patent application Ser. No. 10/864,075, filed Jun. 8, 2004, assigned to the assignee of the present application and incorporated herein by reference. The distal support catheter system can be delivered either concurrently or sequentially with the advancement of the guidewire device 12 to the target site. The position of the guidewire device 12 and catheter system can be maintained and stabilized while the drive shaft 18 is activated out of the axial lumen 16 of the guidewire device 12.

The guidewire device 12, which is described in more detail in co-pending U.S. patent application Ser. No. 11/236,703, comprises an elongate hollow deflectable guidewire body 38 having a proximal portion, a deflectable distal portion, and a flexible intermediate portion along a length therebetween. The elongate hollow guidewire body 38 removably receives the drive shaft 18 within its axial lumen 16 and is coupled to the handle 20 on the proximal portion. The elongate hollow guidewire body 38 may be composed of a unitary structure, such as a single hypotube, which forms a plurality of sections. In a preferred embodiment, the sections comprise a variety of patterns including a proximal interrupted helical pattern and a distal ribbed pattern 40, 42. The elongate hollow guidewire body 38 may be formed from a variety of materials, including stainless steel, polymer, carbon, or other metal or composite materials. The guidewire body 38 may have an outer diameter in a range from about 0.010 inch to about 0.040 inch, an inner diameter in a range from about 0.005 inch to about 0.036 inch, and a working guidewire length in a range from about 150 cm to about 190 cm, as for example in FIG. 1 the length is illustrated as approximately 160 cm.

Referring back to FIG. 2, an exploded view of the distal end portion of the guidewire device 12 shows the drive shaft 18, a tapered pull tube 44 for deflection via the deflection wheel 24, and a radiopaque coil 46 for aid in viewing under fluoroscopy, which are all disposed within the axial lumen 16 of the elongate hollow guidewire body 38. The drive shaft 18 may be movably or fixedly disposed at the distal end of the elongate hollow guidewire body 38. A distal tip 48 of the drive shaft 18 extends distally of the distal end of the hollow guidewire body 38. Upon activation, the distal tip 48 of the drive shaft 18 creates a passageway or enlarges a passageway through the occlusion or stenosis within the vessel lumen. Generally, the distal tip 48 of the drive shaft 18 creates a path at least as large as a perimeter of a distal end of the hollow guidewire body 38. However, it will be appreciated that the path can also have the same perimeter or smaller perimeter than the distal end of the hollow guidewire body 38.

The drive shaft 18 in this embodiment preferably comprises an oscillatory core element, as depicted by arrow 50, which is movably receivable within the axial lumen 16. The preferred oscillating operating mode 50 is of particular benefit as it prevents tissue from wrapping around the distal tip 48 of the drive shaft 18. This in turn allows for enhanced penetration through, in, and/or out of the occlusive or stenotic material. Typically, the drive shaft 18 may be oscillated so that it changes polarity after a period of time. The period of time may in a range from about 0.2 seconds to about 5.0 seconds, preferably in a range from about 0.3 seconds to 1.2 seconds, and more preferably in a range of about 0.7 seconds. Oscillations may be in a range from about 3,600 degrees to about 360,000 degrees.

The drive shaft 18 may additionally comprise an axially translatable drive shaft as depicted by arrow 52 for axial or reciprocation movement so as to completely cross an occlusion. Oscillation movement 50 and reciprocation movement 52 of the drive shaft 18 may be carried out sequentially or simultaneously. Generally, oscillation and/or reciprocation 50, 52 movement of the drive shaft 18 are carried out by a drive motor within the guidewire handle 20, which is described in more detail below. Alternatively, the physician may also manually oscillate and/or reciprocation the drive shaft 18. Additionally, the movable drive shaft 18 may be extended from a retracted configuration to an extended configuration relative to the distal portion of the hollow guidewire body 38, wherein the drive shaft 18 is simultaneously or sequentially extended and oscillated.

The drive shaft 18 may be formed from a variety of materials, including nitinol, stainless steel, platinum iridium, and like materials and have a diameter in a range from about 0.003 inch to about 0.036 inch and a working length in a range from about 150 cm to about 190 cm. The drive shaft distal tip 48 will preferably have an outer perimeter which is equal to or larger than a diameter of the hollow guidewire body 38 so as to create a path at least as large as a perimeter of the distal end of the guidewire body 38. As can be appreciated, the diameter of the drive shaft 18 will depend on the dimension of the inner lumen 16 of the hollow guidewire body 38, the pull tube 44, and/or the radiopaque coil 46.

As mentioned above, the hollow guidewire device 12 of the present invention has a steerability, deflectability, flexibility, pushability, and torqueability which allows it to be positioned through the tortuous blood vessel. Once properly positioned adjacent the occlusion or stenosis, the distal tip 48 of the drive shaft 18 is oscillated and simultaneously or sequentially advanced into the occlusion or stenosis in the vessel lumen to create a path in the occlusion or stenosis. It will be appreciated that the hollow guidewire body 38 and/or the drive shaft 18 may be advanced to create a path through the occlusion or stenosis. For example, once the hollow guidewire body 38 has reached the occlusion, the guidewire 38 together with the oscillating drive shaft 18 may be advanced into the occlusion. Alternatively, the guidewire 38 may be in a fixed position and only the oscillating drive shaft 18 may be advanced into the occlusion.

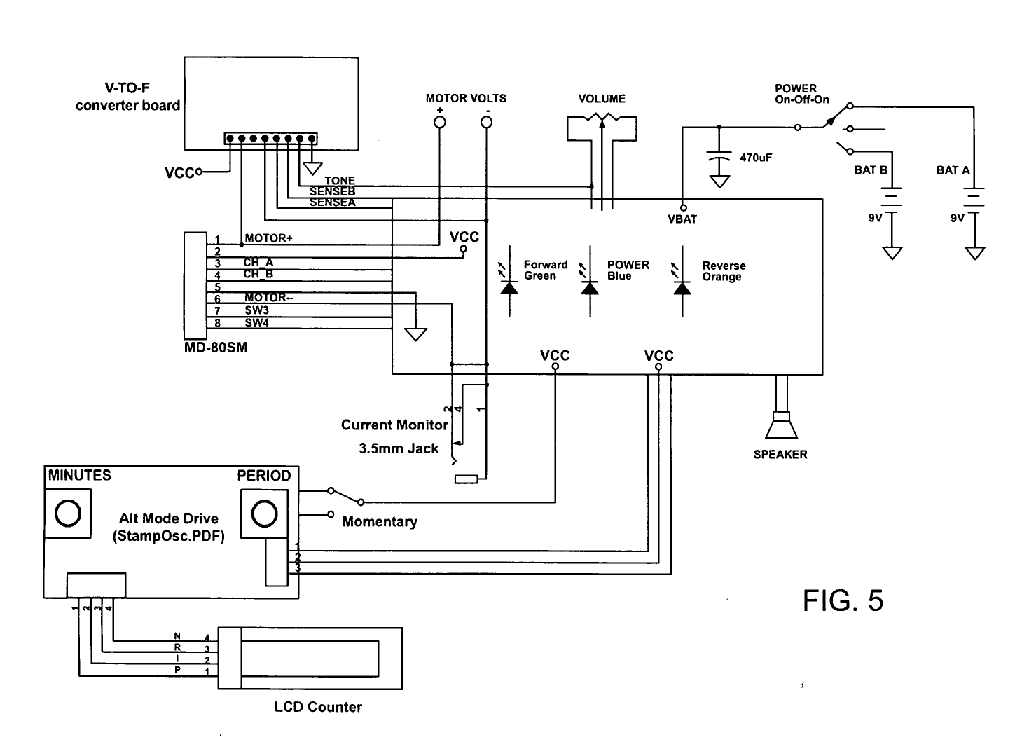

Referring now to FIG. 3, a simplified block diagram of the controller system 10 of the present invention is illustrated. The control unit 14 has a processor 54 which produces a variable sound in response to a load measurement (e.g., resistance encountered) on the drive shaft 18, particularly during advancement of the distal tip 48 in the occluded vessel lumen. The load or resistance encountered may be measured by a change in current in a motor 56 which drives the shaft 18. The motor 56 preferably resides within a distal end of the guidewire handle 20 and is mechanically attachable to a proximal end of the drive shaft 18 to move (e.g., oscillate, axially translate, reciprocate, rotate, vibrate) the drive shaft 18 and distal tip 48. Typically, the drive motor 56 is electrically coupled to the processor 54 via the wire leads or cables 26.

The electronic circuitry in the control unit 14, as for example the oscillation system 58, controls activation of the motor 56 to oscillate (e.g., polarity, period, time), axially translate, reciprocate, rotate and/or vibrate the drive shaft 18. For example, the oscillation system 58 may control the output of the oscillation mode 50 of ± and ± at 0.7 seconds in each direction of the oscillatory drive shaft 18. This output mode may be provided by activation of the momentary switch 34. As another example, the oscillation system 58 may measure the accumulated oscillation time. In this instance, the guidewire device 12 may be automatically disabled once the accumulated oscillation time has exceeded a time threshold in the range from about 60 seconds to about 1,200 seconds, as for example 600 seconds. The accumulated oscillation time may be constantly displayed on the LCD display 36 on the control unit 14. The next oscillation interval may be initiated by turning the main power switch 32 off and then back on.

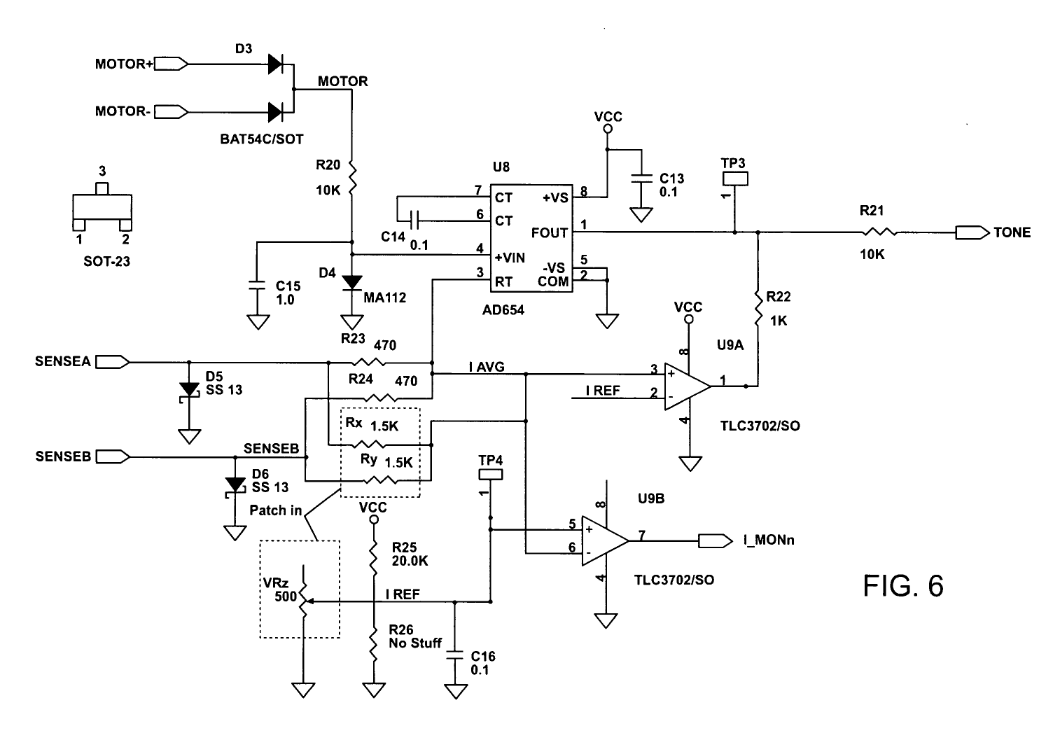

Referring now to FIG. 4, the processor 54 in the control unit 14 further measures loads and produces variable sounds associated therewith after activating the drive shaft 18 as depicted by block 60. Measuring a load comprises measuring a change in current in the motor 56 which drives the shaft 18. Typically, the load on the drive motor 56 varies according to the resistance encountered by the drive shaft 18, as for example hard or soft stenosis in the vessel lumen. The load on the motor 56 may be measured through the detection of voltage across a known resistor, as depicted by block 62, which is directly proportional to the current flowing through the resistor. For example, two 1 ohm resistors may be provided for an oscillatory drive shaft 18, one resistor for each direction of the oscillatory drive motor 56. The amperage related voltage is then compared to a reference voltage, as for example 0.53 volts, as depicted by block 64.

As depicted by block 66, variable sound is then produced by converting the change in current (i.e., the difference between the measured load and the reference voltage) to a frequency for sound via a voltage to frequency generator 68 (FIG. 3). The sound comprises a pitch or tone which is substantially linearly proportional to the measured load on the motor 56. For example, the larger the difference between the measured load and the reference voltage, the larger the change in the pitch or tone of the sound. The different audible tones of the sound are noticeable due the change of the frequency of the signal generated by change in the electrical signal of the load measurement.

The sound may indicate a variety of load conditions within the vessel lumen. In particular, the control unit 14 may provide higher to lower pitches depending on the resistance encountered by the motor 56 through the drive shaft distal tip 48. For example, the variable sound may have a low pitch so as to indicate that the drive shaft distal tip 48 is encountering low resistance which in turn indicates a soft calcification inside the vessel lumen. Alternatively, the variable sound may have a decreasing pitch or even no pitch (e.g., no sound) so as to indicate that the guidewire device 12 is outside the vessel lumen and within sub-intimal tissue. In this instance, the drive shaft distal tip 48 encounters much less resistance outside the vessel lumen as opposed to inside the vessel lumen. Accordingly, the sound emitted may have a much lower intensity or may not even be noticeably present. Still further, the variable sound may have a constant pitch that indicates that the guidewire device 12 is non-operational. In this instance, the zero or no load measurement may indicate a break or fracture in the drive shaft 18 resulting in no change in the sound being emitted so all that is audible are the clicks as the motor 56 changes direction for an oscillatory drive shaft 18. The processor 54 in the control unit 14 may further automatically disable the guidewire device 12 in this situation for safety purposes.

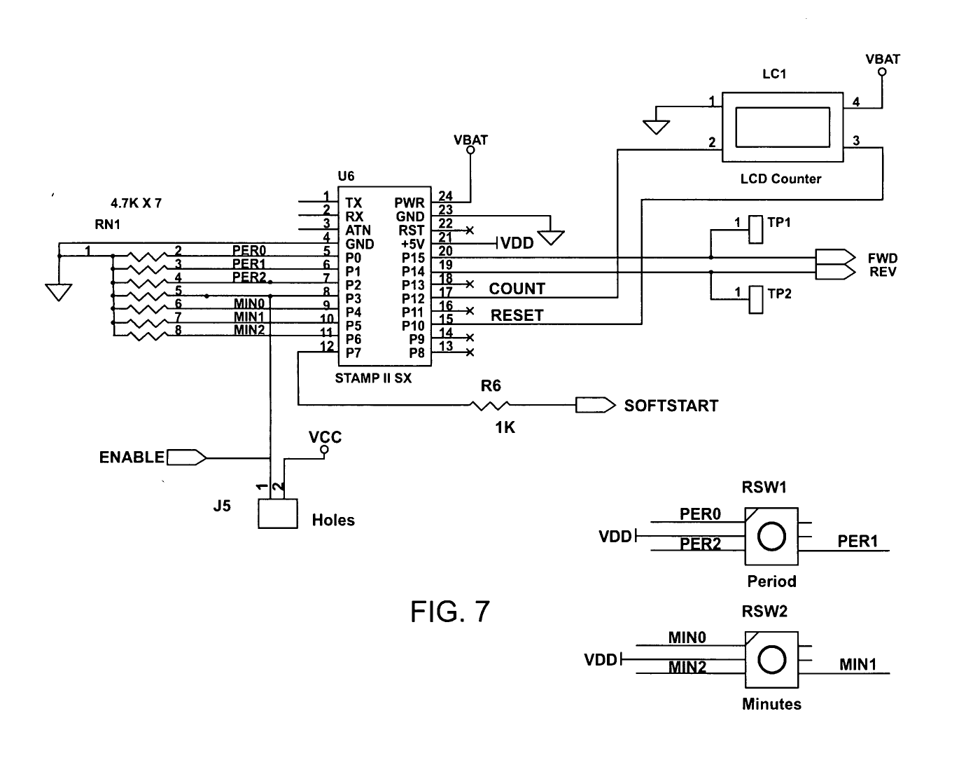

FIGS. 5 through 7 show exemplary electronic circuit diagrams of a circuitry implementation that can be used within the control unit 14 of the present invention. It is understood that many other circuit implementations can be used and yet still arrive at embodiments of the invention. FIG. 5 illustrates various components of the control unit 14 including the processing units 58, 68 described above, the speaker 28 for emitting sound, the volume control knob 30 for adjusting amplification of the audio amplifier 70 (FIG. 3), and the LCD counter 36. Many commercially available processors 54 including 20 MHz processors commercially available from Microchip Inc. may be used. The control unit 14 may be powered by two 9V alkaline batteries via the main on/off power supply switch 32. The power supply may further include a voltage regulator which allows for adjustment of optimum motor 56 speed and torque. It will further be appreciated that the control unit 14 may alternatively be powered via voice activation, wireless activation, or Bluetooth® footswitch technology in lieu of manual activation with switch 32. FIG. 6 illustrates a sample circuit of the voltage to frequency generator 68 which is commercially available from Analog Devices of Norwood, Mass., the workings of which are described in more detail in product description AD654, entitled “Low Cost Monolithic Voltage-to-Frequency Converter,” the full disclosure of which is incorporated herein in by reference. FIG. 7 illustrates a sample oscillation period control and shutdown circuit of the oscillation system 58.

Although certain exemplary embodiments and methods have been described in some detail, for clarity of understanding and by way of example, it will be apparent from the foregoing disclosure to those skilled in the art that variations, modifications, changes, and adaptations of such embodiments and methods may be made without departing from the true spirit and scope of the invention. For example, as described above, another way to measure load is by reading the rotational speed (e.g., rotations per minute) of the drive shaft using an encoder within the drive motor. Basically, the encoder reads the number of revolutions that the drive shaft is rotating at and when any load is sensed then sound changes proportionally. Therefore, the above description should not be taken as limiting the scope of the invention which is defined by the appended claims.

What is claimed is:

1. A guidewire controller system comprising:
a guidewire device having an axial lumen and a drive shaft extending through the axial lumen; and
a control unit coupled to the guidewire device, the control unit having a processor which produces a variable sound in response to a load measurement on the drive shaft.

2. The system of claim 1, wherein the load measurement comprises a change in current or rotational speed in a motor which drives the shaft.

3. The system of claim 2, wherein the change in current or rotational speed is converted to a frequency for variable sound.

4. The system of claim 3, wherein the sound comprises a pitch or tone which is proportional to the measured load on the motor.

5. The system of claim 3, wherein a relationship between the sound and the load measurement is substantially linear.

6. The system of claim 1, further comprising a speaker coupled to the processor.

7. The system of claim 6, further comprising an audio amplifier coupled between the processor and the speaker.

8. The system of claim 1, wherein the guidewire device comprises an elongate hollow deflectable body.

9. The system of claim 1, wherein the drive shaft comprises an oscillatory core element.

10. The system of claim 1, further comprising a handle coupled to a proximal end of the guidewire device.

11. The system of claim 10, wherein the control unit is positioned within the handle.

12. A method for providing control feedback during crossing of an occlusion or stenosis within a vessel lumen:
positioning a guidewire device into the vessel lumen adjacent the occlusion or stenosis;
activating a drive shaft within an axial lumen of the guidewire device;
measuring a load on the drive shaft; and
producing a variable sound in response to the load measurement.

13. The method of claim 12, wherein measuring a load comprises measuring a change in current or rotational speed in a motor which drives the shaft.

14. The method of claim 13, wherein producing a variable sound comprises converting the change in current or rotational speed to a frequency for sound.

15. The method of claim 14, wherein the sound comprises a pitch or tone which is substantially linearly proportional to the measured load on the motor.

16. The method of claim 14, wherein the variable sound indicates a level of occlusion in the vessel lumen.

17. The method of claim 14, wherein the variable sound indicates that the guidewire device is outside the vessel lumen and within sub-intimal tissue.

18. The method of claim 14, wherein the variable sound indicates that the guidewire device is non-operational.

19. The method of claim 18, further comprising automatically disabling the guidewire device.

20. The method of claim 12, further comprising emitting the sound from a speaker.

21. The method of claim 20, further comprising amplifying the sound prior to emission from the speaker.

22. The method of claim 12, wherein activating comprises oscillating the drive shaft.

23. The method of claim 22, wherein oscillating the drive shaft comprises changing polarity after a period of time in a range from 0.3 seconds to 1.2 seconds.

24. The method of claim 23, wherein the time period comprises 0.7 seconds.

25. The method of claim 23, wherein the activating, measuring, producing, and changing polarity steps are carried out by a processor.
